(19) United States
(12) Patent Application Publication
(10) Pub. No.: US 2004/0023299 A1
(43) Pub. Date: Feb. 5, 2004

(54) Diagnosis of liver disease

(76) Inventors: Rowan James Milner, Gainesville, FL (US); Schalk Willem Van Der Merwe, Pretoria (ZA)

Correspondence Address: COZEN O' CONNOR, P.C., 1900 MARKET STREET, PHILADELPHIA, PA, 19103-3508, US

(21) Appl. No.: 10/343,090

(22) PCT Filed: Jul. 23, 2001
(86) PCT No.: PCT/GB01/03304

(30) Foreign Application Priority Data
Jul. 24, 2000 (ZA) 2000/3726

(51) Int. Cl. A61K 51/00; G01N 33/53

(52) U.S. Cl. 435/7.1; 424/1.49

(57) ABSTRACT

Hepatic fibrosis or necrosis is diagnosed in vivo by administering a complex of a polymer having calcium-chelating phosphonate groups with radionuclide ions and comparing the degree of retention of the polymer in the diseased liver with that of normal patients. The preferred complex is of Tc-99m polyethyleneiminomethyl phosphonate.

### BACKGROUND OF THE INVENTION

1. Field of the Invention

This invention relates to the use of a polymer formulation for in vivo diagnosis of liver fibrosis and necrosis.

2. Description of the Related Art

Hepatic fibrosis is the accumulation of fibrous extra-cellular matrix or scar tissue in patients with chronic liver injury. Left untreated, it progresses to cirrhosis, which is at present incurable. The chronology of events is as follows:

(1) damage to the liver, e.g. by toxic metabolites;

(2) necrosis (cell death);

(3) inflammation;

(4) formation of fibrous extracellular matrix (fibrotic tissue).

The main cause of hepatic fibrosis is activation of hepatic stellate cells: see the review by Scott L. Friedman, “Cytokines and Fibrogenesis”, Seminars in Liver Disease” 19 (2), 129-140 (1999). These cells become activated by injury or toxic insult to the liver, setting in train the above chain of events. The review proposes that this process may be arrestable by modulating the action of certain cytokines. With this background in mind, it would be desirable to diagnosis hepatic fibrosis or the necrosis which precedes it at an early stage.

Technetium-99m (the “m” refers to the metastable state of this particular form of the isotope) is a gamma-ray emitter. Tc-99m phosphates were introduced for bone imaging 30 years ago and there have been many reports of deposition of the Tc in soft tissue, including the liver: see, for example, the summary contained in Amilcare Gentili et al., “Nonosseous accumulation of bone-seeking radiopharmaceuticals”, Radiographics 10, 871-881 (1990). This paper mentions that uptake by the liver is frequently caused by metatstatic deposits from various carcinomas of other organs. In relation to the heart, it refers to the use of Tc-99m pyrophosphate to detect acute myocardial infarction. It comments that the findings from gamma ray scintigraphy with this reagent are similar to those observed with use of Tc-99m diphosphonates, when there is an excess of tissue calcium, following local tissue necrosis or damage.

It is stated in Kenneth P. Lyons et al., “Localization of Tc-99m pyrophosphate in the liver due to massive liver necrosis: Case report”, Journal of Nuclear Medicine 18 (6), 551-552 (1977) that technetium labelled phosphates localise in organs with cellular injury and necrosis. The paper refers to the possible diagnostic benefit of the Tc-labelled phosphate in patients presenting with liver damage from alcohol, hepatitis, hepatotoxicity and passive congestion.

Barry M. Flynn and S. T. Treves, “Diffuse hepatic uptake of technetium-99m methylene diphosphonate in a patient receiving high dose methotrexate”, The Journal of Nuclear Medicine 28 (4), 532-534 (1987), describe hepatic uptake of technetium-99m as an unusual phenomenon which has been reported in hepatic metastases, hypercalcemic states, hepatic necrosis and amyloidosis. The paper reported a case study of severe, reversible hepatic dysfunction, apparently caused by methotrexate toxicity. The uptake of technetium-99m methylene diphosphonate in the patient's liver was revealed after a bone scan.

Wei-Jen Shih and John Coupal, “Diffuse and intense Tc-99m HMDP localization in the liver due to hypoxia secondary to respiratory failure”, Clinical Nuclear Medicine 19 (2), 116-120 (1994) reported a case of respiratory distress syndrome, following a lobectomy of the lung to remove a carcinoma. A Tc-99m hydroxymethylene diphosphonate bone scan revealed intense hepatic uptake, interpreted to indicate hepatic necrosis.

C. H. Evans and D. C. Mears, “Binding of the bone-seeking agent 99m Tc-1-hydroxyethylidene-1,1-diphosphonic acid to cartilage and collagen in vitro and its stimulation by Er³⁺ and low pH”, Calcified Tissue International 32, 91-94 (1980), suggest that collagen sequesters this compound and contributes to its uptake by cartilage and bone.

Other bone-seeking Tc-99m reagents are known, including “Polymin-MP”, which is polyethyleniminomethyl phosphonic acid, see W. Louw et al., Biodistribution of radiolabelled Polymin-MP of different molecular sizes as a selective bone-seeker for therapy in animal models”, European Journal of Nuclear Medicine 25 (8), 1167, Abstract PS-733, (1998) and R. J. Milner et al., “The biodistribution, pharmacokinetics, bone localisation of variously molecular sized molecular radiolabelled Polymin-MP in normal dogs and dogs with osteosarcoma of the appendicular skeleton”, European Journal of Nuclear Medicine 26 (9), 1220, Abstract PS-669, (1999).

### SUMMARY OF THE INVENTION

It has now surprisingly been found that polymers having free phosphonate groups positioned to provide polydentate ligands capable of forming stable complexes with radionuclide ions and capable of binding calcium ions, are useful in the preparation of complexes with radionuclide ions, which, when administered to the patient, enable hepatic fibrosis or necrosis to be diagnosed satisfactorily, said polymer having a molecular weight which enables uptake into the liver, retention by a diseased (fibrotic or necrotic) liver for sufficient time reliably to count the radioactivity in the liver so that it can be compared with a normal liver and sufficiently rapid clearance by the kidneys to prevent long-term harm to the patient.

The principles on which the invention is based comprise the following features:

1. The free phosphonate groups provide a calcium chelating function in vivo. It appears that the onset of fibrosis is associated with the accumulation of calcium ions, which are chelated by the polymer, causing the polymer to be retained within the diseased liver for a longer time than in a healthy liver.

2. The binding of the calcium ions to the phosphonate groups is associated with the presence of collagen in the hepatic fibrous tissue.

3. The phosphonate groups enable a stable complex to be formed initially with radionuclide ions. Possibly, subsequent binding of the polymer by calcium in the diseased liver displaces the radionuclide ions, but, anyhow, they are sufficiently retained by the liver for diagnosis to take place. Ultimately they pass out of the liver for clearance by the kidneys. In the normal liver the degree of retention is very small.

4. By providing a large number of (anionic) phosphonate groups in the polymer, the polymer as a whole is rendered considerably anionic, so that it does not enter cells. This assists in ensuring that sufficient polymer is available for interaction with the fibrous extra cellular matrix, thus giving scans with a low background which will enhance imaging.

5. The relative molecular mass of the polymer is selected so as to ensure that it will enter the liver and be released therefrom after diagnosis, and so that (after retention by the diseased liver) it will be cleared rapidly from the body through the kidneys. It will normally be at least 5 kiloDaltons (kDa).

The term “rapid clearance” as used herein means sufficiently rapid to avoid long-term radiation damage to the patient. As a guide, it will usually mean that when the radiolabelled polymer is given to healthy male baboons, the half life of the radioactivity in the liver is less than 45 minutes.

The formulation administered is a radionuclide complex of the polymer, which will normally be freshly prepared for administration to the patient. This complex is not in itself novel, since the preferred embodiment thereof, namely Tc-99m polyethyleniminemethyl phosphonate, abbreviatable to “PEIMP”, has been previously disclosed for bone imaging: R. J. Milner et al. and W. Louw et al., above. Consequently, the invention comprises a second in vivo diagnostic use of this active principle and is sought to be protected in whatever manner is allowable according to the patent laws of the designated states.

The term “diagnosis” is used herein generically to refer to any qualitative, quantitative or semi-quantitative determination of the existence of the disease or the extent of its progression (prognosis) and is to be construed liberally accordingly.

Wherever patent law permits, in particular in Australia and the USA, the invention provides a method of in vivo diagnosis of hepatic fibrosis or necrosis in a patient, which comprises administering parenterally to the patient an effective amount of a complex of (1) a polymer as defined above and (2) radionuclide ions which are non-toxic in the diagnosis, determining the degree of the retention of the radionuclide within the liver of the patient under diagnosis, relative to that of normal patients, and from the result of this determination making a diagnosis as to whether the patient is suffering from hepatic fibrosis or necrosis or the extent thereof.

Wherever patent law permits, in particular in Europe, the invention provides the use of the polymer defined above in the preparation of a formulation thereof as a complex with radionuclide ions as defined above, for the in vivo diagnosis of hepatic fibrosis or necrosis or the extent thereof.

Wherever patent law permits, in particular in Japan and South Korea, the invention provides a composition comprising the said complex, for the said purpose.

It is contemplated that the unauthorised seller of the polymer for the purpose of making up a complex to be administered for the purpose of diagnosis of hepatic fibrosis or necrosis of the liver will be a contributory or indirect infringer of the patent to be granted on the present application and such persons are hereby put on specific notice to that effect.

The term “complex” is used herein to include any form of complex-forming co-assembly of the polymer and radionuclide ions. Thus, it includes mixtures of the polymer with a radionuclide salt and, if necessary, a reducing agent to reduce the radionuclide to an appropriate oxidation state. For example, it includes a mixture of the polymer with a radioactive technetium as its pertechnetate salt and stannous chloride as a reducing agent.

### DESCRIPTION OF THE PREFERRED EMBODIMENTS

The Polymer

The kind of polymer is not critical provided that it carries phosphonate groups available for polydentate ligand formation and can be formulated for convenient administration (preferably so that it is dispersible or soluble in water).

Preferably the polymer is a polyamine. The term “polyamine” as used herein refers to any polymer of the requisite relative molecular mass which carries a multiplicity of free amino groups, whether or not its main chain is formed from an imine or amine. Thus, it includes polymers which have no nitrogen atoms in the main chain but have amino groups subtended therefrom.

The polymer can be straight or branched in its main chain and may be a dendrimer. Dendrimeric polymers are macromolecules which have a progressively branched structure, whereby branch is formed upon branch. The term comes from the Greek “dendra” (tree), but in the context here this does not imply that the polymer must have a straight “trunk” as does a tree. Dendrimers may be prepared from a monomer which undergoes branching reactions during polymerisation, whereby a second branch extends out of the first, a third branch out of the second and so on (although not every branch is necessarily itself branched). The polymer is preferably a dendrimeric polyamine, most especially a homopolymer or copolymer of ethylene imine. Ethylene imine forms dendrimeric addition polymers with itself, which have the partial, illustrative schematic formula:

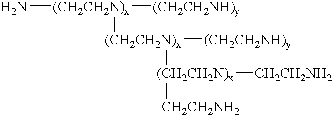

The symbols “x” and “y” represent schematically variable numbers of units interspersed along the chains and the primary, secondary and tertiary amine units are substantially randomly dispersed within the polymeric structure. Typically there are about 50% primary and 25% each of secondary and tertiary amine groups.

The polymer may be a random, block or graft copolymer. Thus, an ethylene imine polymer may contain other units than ethylene imine, e.g. ethylene oxide, propylene oxide or acrylic or methacrylic monomers, e.g. acrylamide or 2-N,N-diethylaminoethyl acrylate or the corresponding methacrylates. However, the ethyleneimine polymer must remain reasonably well branched, to allow a multiplicity pf phosphonate groups to be attached to primary amine groups at the ends of the chains and thereby provide a strong chelating function.

Alternatively, the polyamine may be non-dendrimeric. For example, a chloromethylpolystyrene starting polymer can be aminated in the position of chloromethyl-substitution, e.g. para-position or ortho- and para-positions, to give an aminomethylpolystyrene and this can then be reacted with formaldehyde and phosphorous acid to give the methylphosphonate(phosphonomethylpolystryene).

The term “phosphonate group” as used herein includes bisphosphonates. Generally, the phosphonates are of formula —R—PO(OH)₂)ₙ where n is from 1 to 3 and R is an organic group of valency to match the value of n, directly or indirectly attached to a chain of the polymer. R may be aliphatic, cycloaliphatic, aromatic, araliphatic etc. Preferably it is the residue of an alkane, hydroxy-substituted-alkane or benzene. Any remaining valencies are satisfied by hydrogen atoms or substituents. In bisphosphonates, n=2. Preferably n is 1 or 2. When n=1, the organic group R is preferably methylene, 1,1-ethylene, 1,2-ethylene, 1-hydroxyethylene, 1,3-, 1,2- or 2,2-propylene or p-phenylene or p-tolylene. When n=2, the same R groups, but with another —PO(OH)₂ group replacing a terminal H-atom (where possible), are preferred.

The phosphonate groups may be arranged on the polymer in any way effective to provide at least two neighbouring groups per cation bound. In other words, the ligand provided by the polymer must be at least bidentate. It may have a higher degree of dentation than 2. The phosphonate groups may be geminal (attached to the same carbon atom) as in, for example, polystyrylpropane-2,2-bis-phosphonates having repeating units of formula

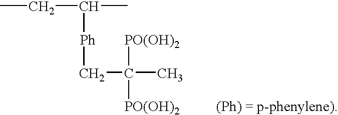

See Mats J. Sundell and Jan H. Näsman, “Desk Reference of Functional Polymers, Syntheses and Applications”, American Chemical Society (1997), Chapter 1.8, lines 120-126 at page 120. Alternatively, the phosphonic acid groups may be direct-vicinal (attached directly to adjacent C-atoms) or near-vicinal (attached to alternate carbon atoms) as in e.g. polyvinylphosphonic acid. Another form of attachment is pendant-vicinal or pendant-near-vicinal, being dependent from, but not attached directly to adjacent or alternate carbon atoms as in e.g. PEIMP or in polystyrylphosphonates having repeating units of formula

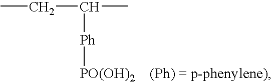

the monoethyl ether of which is described by Mats J. Sundell and Jan H Näsman above, at page 126.

The phosphonate groups attached to the primary amine groups of a polyamine, e.g. PEIMP, are preferably of formula —R′—PO(OH)₂, wherein R′ is an alkylene group of 1 to 4 carbon atoms, which may have a branched chain, preferably methylene, 1,2-ethylene or 1,1-ethylene. The alkylene group may optionally be hydroxy-substituted, especially in the α-carbon atom (adjacent to the P-atom).

Another type of polymer usable in the invention is a polyethylene-vinylbenzene graft copolymer containing bisphosphonate groups, which may be prepared as described by Mats J. Sundell and Jan H. Näsman, above, at page 127.

Other usable polymers include hydroxymethylphosphonic or 2-ethylphosphonic esters of acrylic or methacrylic acid, optionally copolymerised with acrylic or methacrylic acid.

Persons skilled in the polymer art will appreciate that it is desirable to formulate these polymers as injectible solutions or suspensions, preferably aqueous. Therefore, it may be desirable to copolymerise the relevant monomers with co-monomers having functionalities which improve the solubility of the homopolymer, especially water-solubility. Thus co-monomers bearing carboxylic acid groups will usually be particularly appropriate.

Preparation of phosphonates from polyamines

Phosphonate groups may be added to polyamines, e.g. ethylene imine polymers, by the steps of condensing the polyamine with an aldehyde and phosphorous acid or a suitable derivative of phosphorous acid. The starting polyamine may have a molecular mass range outside that finally required, since the end product can easily be size-separated. For example, an ethylene imine polymer may have a size range of 2-100 kDa. The aldehyde may be formaldehyde, for example as an aqueous solution thereof and the derivative of phosphorous acid may be a salt of phosphorous acid. The polymer may thus be polyethyleniminomethyl phosphonic acid (PEIMP) or a salt thereof.

A similar reaction can be carried out on other polymers having pendant primary amino groups.

The preferred method of preparation comprises heating an aqueous solution of phosphorous acid or a derivative thereof to its reflux temperature in the presence of a strong acid such as hydrochloric acid, slowly adding the aldehyde and the polymer, in any sequence but in a pre-selected molar ratio, to the aqueous solution, heating the mixture until the reaction has progressed to a sufficient degree and isolating a fraction having a molecular mass range of 10-30 kDa, more preferably 20-30 kDa, e.g. by membrane ultrafiltration.

Relative Molecular Mass

The optimal r.m.m. of the polymer is governed solely by the in vivo biological requirements referred to above. When the r.m.m. is too high or too low, the polymer is cleared less rapidly from the kidneys, so the limits can be determined experimentally in small animals. The term “rapid clearance” usually signifies that the half life of the radioisotope in the liver of healthy male baboons is less than 45 minutes, but preferably it should be less than 30 minutes. In the unlikely event of any ambiguity arising from this preferred definition, the procedure and baboons of Example 2 shall be definitive in setting the conditions of this test. The exact numerical limits of the r.m.m. are not sharp and will depend on the particular polymer. It is unlikely to be less than 5 kDa and more usually will be at least 10 kDa. If PEIMP is used, it will normally have a relative molecular mass of about 10 kDa to about 30 kDa.

All r.m.ms herein referred to are those obtainable by ultrafiltration (or gel permeation chromatography) on membranes, especially polyethersulfone membranes. However, the r.m.m. may be determined in other ways which give equivalent values, e.g. by viscosity measurement. Radionuclide ions are excluded from the r.m.m. figures herein.

The Radionuclide

The radionuclide may be any tolerable for the purpose, having a half life adequate for the diagnosis to take place, but preferably as short as possible. Preferably it is technetium-99m which is routinely used in nuclear medicine for diagnostic purposes. Technetium-99m has a half life of about 6 hours. It decays to emit gamma rays and low energy electrons. Generators of Tc-99m are commercially available. They are based on Mo-99 which decays to Tc-99m. The generators have a short shelf-life (about 2 weeks). Tc-99m is not to be confused with Tc-99 obtained from nuclear re-processing, having a half life of 2.13×10⁵ years. Other suitable radionuclides for use in the invention will be apparent to those skilled in the radiochemicals field, especially indium-111, gallium-67, gallium-68 and tin 117m.

To form a complex of the radionuclide and the polymer as hereinbefore described, a radionuclide salt may be dissolved in water and the polymer dispersed or dissolved in finely divided form in the water at a pH of typically 4-7. The pH will be selected to promote dissolution in the water of the compound containing the radionuclide and to promote formation of the complex from the radionuclide and the polymer. The metal species of the radionuclide may be already in a suitable oxidation state for complexation with the polymer or it may be in a higher oxidation state, in which case the reaction is conducted in the presence of a reducing agent to lower the oxidation state of the radionuclide so that it will complex with the polymer. For example, the radionuclide salt may be a pertechnetate and the reducing agent may be a stannous salt such as stannous chloride or stannous chloride dihydrate. However, the indium and gallium salts can be supplied simply as non-toxic cationic salts such as chlorides, no reduction being thus required.

pH Adjustment and Salts

The method may include the step of adjusting the pH of the reaction mixture after formation of the complex to a physiologically acceptable value. The pH may be adjusted with a physiologically acceptable acid or alkali which does not interfere with the formation of the complex. For example, the pH may be adjusted with an acid such as hydrochloric acid or acetic acid, or with a base such as the hydroxide, carbonate or bicarbonate of an alkali metal, aqueous ammonia or an ammonium salt such as ammonium carbonate or bicarbonate. The alkali metal may conveniently be sodium or potassium. Most preferably, a sodium salt will be employed, for reasons of non-toxicity and water-solubility.

Administration

The complex is administered to the patient in any way appropriate to penetrate rapidly to the liver. Given that it is desirable to use a radionuclide of relatively short half life, only methods which ensure fast entry into the circulation will be of interest, principally injection and most usually i.v. injection. Thus, the complex will normally be made up as a sterile injectible solution or dispersion, in any non-toxic solvent, but usually aqueous.

The amount of radionuclide administered will depend on its half-life. It will typically be in the range 3.5 to 15 MBq, more usually 3.7 to 10.4 MBq Tc-99 m/kg body weight of the patient. Equivalent dosages can be estimated for other radiosotopes.

Determination of Degree of Retention of the Radionuclide in the Liver

Any method can be used which directly or indirectly enables a comparison to be made between the retention of the polymer in the normal and diseased liver. Conveniently, a gamma-emitting isotope will be used and gamma ray scintigraphy employed. One preferred method makes use of (a) a static image early in the study to detect or assay necrosis and (b) a static image later in the study to detect or assay fibrosis. These may conveniently be taken at (a) 20-40, preferably 30 minutes and (b) 2-4, preferably 3 hours. The lengths of time over which the images are taken may be, for example 1 minute and 2 minutes respectively. The determination may be made in various ways, especially by measuring a liver: cardiac blood pool ratio. A ratio higher than normal indicates fibrosis or necrosis. It is also possible to determine the percentage of the amount initially injected present in the liver. This will be significantly higher in the diseased liver. This will be significantly higher in the diseased liver. Variations of these methods can easily be devised. This will be significantly higher in the diseased liver. The method of the invention can be made accurate enough to quantify or semi-quantify the state of the disease, which is useful in monitoring the effect of patients of treatments to ameliorate, their condition. Likewise, it is helpful in model animals to test the efficacy of candidate drugs or regimes of treatment.

The invention is illustrated by the following non-limiting Examples. Tables are shown at the end of the description. Radiation doses have been converted from milliCuries (mCi) to megaBecquerels at 1 Curie=3.7×10¹⁰ Becquerels, which is equivalent to 1 milliCurie=37 megaBecquerels. T½ values given in the Tables refer to half the time which it would take to clear the radioactivity from the specified organ, based on time plots, (and are, of course, not T½ for the isotope which is a constant).

### EXAMPLE 1

1. Synthesis of Polyethyleniminomethyl Phosphonic Acid (PEIMP)

Phosphorous acid (18.36 g) and concentrated hydrochloric acid (51.3 ml) were added to a reaction vessel equipped with a thermometer, magnetic stirrer bar, dropping funnel and condenser under an atmosphere of argon. Dissolution of the phosphorous acid was achieved by stirring and heating to 80° C. The dropping funnel was charged with formaldehyde solution (23.3 ml) and the solution was added dropwise to the phosphorous acid. The temperature was raised to reflux (90° C.) and a solution of polyethylenimine (8.33 g in 40 ml water) was slowly added at a rate of 0.3 ml/min with the aid of a peristaltic pump to form a reaction mixture. The reaction mixture was continuously purged with argon. When addition of the polyethylenimine solution was completed, the reaction mixture was stirred under reflux at 90° C. for a further hour, then allowed to cool slowly overnight, during which time a product separated as a viscous oil. The mother liquor was decanted, water (50 ml) was added and the product formed a doughy mass and a liquid phase upon stirring.

The liquid phase was decanted and the water addition, stirring and the decanting were repeated twice more. The doughy material was dissolved in 1M sodium carbonate solution (36 ml) to form the water-soluble sodium salt of PEIMP at a pH of 7.0. After lyophilization, 12 g of macromolecular sodium PEIMP was obtained.

2. Purification and Fractionation of Sodium PEIMP

Sodium PEIMP, prepared in Section 1, was fractionated by sequential membrane ultrafiltration, using commercially available polyethersulfone membranes, to yield various fractions of various relative molecular masses. The membrane retentates were collected, concentrated to a tenth of the volume, diluted with distilled water, re-ultrafiltered and collected again. A typical yield of the 20-30 kDa fraction was 9%.

3. Labelling of PEIMP with Tc-99m

To prepare a kit, lyophilised sodium PEIMP, prepared as described in Section 2, in a sealed vial (5 mg) and a reducing agent (stannous chloride dihydrate, 0.25 mg) was complexed with technetium-99m by adding an aqueous solution, preferably physiological saline, of sodium pertechnetate-99m, at about 10 ml (up to 1850 MBq) to the vial, to produce a technetium-99m labelled complex of the PEIMP (pH 5.0-5.5). Samples of the radiolabelled complex were analysed for radiochemical purity using thin-layer chromatography on silica gel-impregnated glass-fibre sheets as stationary phase and acetone and 0.9% aqueous sodium chloride solutions as mobile phase. The radiochemical purity thereof was >95%, which is well within nuclear medical requirements.

4. General Scintigraphic Procedure

This is a general description which, unless otherwise stated, is not related to the particular species undergoing the diagnosis. Scintigraphy is the procedure used in nuclear medicine whereby a gamma-emitting radioactive substance (tracer) injected intravenously into a patient is followed in its passage through the body of the patient and whereby its sites of accumulation are localised by detecting and counting the radioactive emissions using a NaI-scintillation crystal, array of photomultiplier tubes, and analogue to digital conversion for quantification by a dedicated computer. This instrumentation is called a gamma camera plus data processor. Such cameras are available with single, dual, or triple heads which house the NaI-crystal(s). A dynamic study is a sequence of short duration images acquired by the camera in quick succession, mostly immediately after tracer injection. A static image is an image at a specific time post tracer injection and mostly of longer duration, typically 2 minutes.

A single/dual head gamma camera was positioned to view the organ, e.g. thorax and liver in dogs and baboons. This position would also be usable in humans to view at least the liver and cardiac blood flow. A dose of about 3.7 to 10 MBq/kg body weight of the Tc-99m sodium PEIMP fraction was given. Dynamic and static studies took place as described. The rationale behind the invention is that for necrosis the polymer “tracer” will seek and leave sites of necrotic cells early after its administration to the patient. On the other hand the “tracer” will tend to stay longer at a site of fibrosis, and will therefore predominantly mark fibrosis at the later, say 3 hour, scan.

Processing was carried out to yield (variously) the following information:

Time-activity curves of the cardiac blood pool, liver and kidneys, to display biokinetics

Percentage relative organ distribution of above tracer

Liver: cardiac blood pool count rate ratios vs time, post injection.

### EXAMPLE 2

In vivo Pharmacokinetics and Biodistribution of Tc-99m-PEIMP Fractions of r.m.m. 3-10, 10-30, 30-50, 50-100 and 100-300 kDa in Baboons

Twenty healthy male baboons (Papio ursinus), average weight 27.5 kg, were used in the study and each received i.v. Tc-99m PEIMP of various relative molecular mass ranges. All studies were performed after approval by the Ethics Committee, according to the guidelines of the National Code for Animal Use in Research, Education, Diagnosis and Testing of Drugs and Related Substances in South Africa.

The baboons (n=4 in each group) were subjected to identical experimental procedures except that the mentioned differences in relative molecular mass ranges of the polymer. Five different relative molecular mass ranges of the PEIMP were studied, viz (i) 3-10 kDa, (ii) 10-30 kDa, (iii) 30-50 kDa, (iv) 50-100 kDa and (v) 100-300 kDa. Induction of anaesthesia was performed with ketamine hydrochloride (10 mg/kg i.m.), (Ketalar Parke Davis, Cape Town), and immediately followed by a maintained controlled infusion of a 6% sodium pentobarbitone solution (Sagatal Kyron Laboratories Pty Ltd, Benrose) at 30 ml/h. The animal in the supine position under the gamma camera was injected i.v. with a bolus of 185-259 MBq of Tc-99m PEIMP and data acquisition started on a count down with a Siemens Orbiter gamma camera in 64×64 word mode performing a 30 min dynamic study (30×1 min frames). At one, two, three and four hours, and also at 24 hours static images of 120 sec were acquired.

Blood and urine samples were collected at fixed intervals for four hours, viz every three min for the first hour, then hourly for blood samples, and urine every five min for the first hour, subsequently hourly. The activity and volume of each sample were recorded. These blood samples were taken from an indwelling catheter in the jugular vein.

Regions of interest (ROIs) were placed on the images of cardiac blood pool, liver, lung, spleen, kidneys, and bone (the vertebrae) to obtain time-activity curves of the dynamic study. Similarly, data of count rate per pixel for the ROIs, which were decay-corrected, were obtained from the static images. These were normalised to extend the time-activity curves of the dynamic study to four hours.

Blood clearance and cumulative urine curves were also obtained in all cases so that average relative organ distributions of the retained activity and eventually of the injected dose (i.d.) could be obtained for all molecular size fractions of the polymer. The statistical analysis was performed by Student's t-test for paired variables on a 5% level of confidence. The results appear in Tables 1-3.

To optimise the molecular size of the macromolecule for its selectivity towards diseased liver areas, excessive normal liver uptake should be minimised, also potentially harmful kidney and bone uptake. This would exclude fractions >50 kDa (liver), and also 30-50 kDa, and 3-10 kDa, which leave the kidneys vulnerable to radiation exposure. The kidneys should however be the pathway for excretion. Although uptake by the skeleton is the highest for this fraction, it is still much lower than for most phosphonates. The size range 10-30 kDa, even more specifically 20-30 kDa, also remains in the cardiac blood pool for T½=10 min, long enough time for accumulation by the lesion in the liver.

A study of the in vivo pharmacokinetics and biodistribution of the 20-30 kDa fraction of sodium PEIMP subsequently labelled with technetium-99m in normal healthy dogs showed satisfactory results, reported in Example 6 of the South African priority application, but omitted here, for brevity. The liver to cardiac ratio at various times after administration of the polymer complex ranged from 0.7 to 0.9:1. It remained at less than 1:1 for 3 hours, increasing slightly with time.

### EXAMPLE 3

Biodistribution of the Tc-99m 20-30 kDa Sodium PEIMP in a Dog with Histologically Confirmed Liver Fibrosis

The Tc-99m 20-30 kDa PEIMP was prepared as described in Example 1 and administered at 185 MBq (10 MBq/kg) by bolus injection to a German Shepherd dog with histologically confirmed liver fibrosis. The biodistribution was scintigraphically determined for 3 hours as described in Example 2. The scintillation scan showed a higher uptake of the Tc-99m 20-30 kDa PEIMP in the liver of the affected dog than the control, and a higher ratio liver: cardiac ratio (i.e. 1.11:1 vs. 0.75:1 for controls). This corresponds with faster clearance from the blood pool in the diseased dog than in healthy dogs and slower clearance from the liver. (T½ cardiac) control=19 min vs. (T½ cardiac) sick=12 min. (T½ liver) control=15 min vs. (T½ liver) sick=22 min. Table 4 includes the results.

This result shows that the present invention is useful for the in vivo diagnosis of liver fibrosis or necrosis. In particular the invention is believed to promote acceptable selectivity by inducing diagnostically effective uptake of radionuclides at diseased liver areas in the human or animal body, without simultaneously causing unacceptable radiation damage to other organs, bone and bone marrow, to leave such normal organs and background largely free of radioactivity for better lesion detection, and to remain in the vasculature sufficiently long to allow adequate and reproducible accumulation at the lesion site, which will be quantifiable.

### EXAMPLE 4

Biodistribution of Tc-99m 20-30 kDa Sodium PEIMP in Rat Models of Liver Fibrosis and in Control Rats

The studies were performed after approval by the Ethics Committee of the University of Pretoria, according to the guidelines of the National Code of Animal Use in Research, Education and Testing of Drugs and Related Substances in South Africa. These guidelines accord with international standards.

Adult male rats (Sprague-Dawley), with an average starting weight of 200 g were used for this study. The animals were divided into four groups: 1 control group, consisting of 5 animals and 3 experimental groups, consisting of 8 animals each.

Chronic liver disease was established by exposing the animals of the 3 experimental groups to weekly intra-gastric administration of CCl₄. A starting dose of 80 μl was used and increased weekly, based on the weight of the animals. Nothing was administered to the control group. All the animals were weighed on day one and the experimental groups started with their 80 μl CCl₄. After three days the animals were again weighed to determine the dose for the second week. That was the procedure for the administration of the CC_{4 l during the entire experiment. On day three of week five, the scanning of group} 1 started. Anaesthesia in each case was induced with an intraperitoneal injection of sodium pentobarbitone 6% solution (Sagatal, Kryon Laboratories Pty Ltd., Benrose, Gauteng, S. A). Each rat was positioned on the camera (Siemens Orbiter Gamma Camera) and injected with 37 MBq/kg of Tc-99m 20-30 kDa PEIMP on a countdown for a dynamic, scintigraphic acquisition of data for 15 min. The procedure for group 2 (7 weeks) and group 3 (9 weeks) was the same as for group 1, except that the dynamic scintigraphic acquisition was for 25 min. After the completion of the acquisition, the rats were terminated by an intravenous overdose of sodium pentobarbitone. The livers were taken out, stored in a 10% formalin solution until the next day and then processed and stained with Haematoxylin and Eosin, Reticulin and Cyrus red.

From the dynamic study, time-activity curves were obtained for the cardiac blood pool region, liver and kidneys. Mean values of relative distribution of the radioactivity in various organs were subsequently calculated as a percentage of injected dose (% ID) and percentage of retained activity and compared for the 4 groups. A ratio for liver: cardiac (L/C) activity was also determined and plotted as a function of time. This ratio tracks radioactive uptake in the liver with respect to radioactivity present in the cardiac blood pool.

Increased liver retention time and uptake occurred in the treated rats with respect to controls. This is summarised in Table 5, where T½ for each group is presented with maximum percentage uptake of radioactivity in cardiac blood pool, liver and kidneys. As can be seen, there is increased reduction in T½ of the cardiac blood pool, and prolonged retention in the liver. The L/C ratios, demonstrating abnormally fast clearance from the cardiac blood pool for the treated rats, and an increasing accumulation of radioactivity in the liver, are shown in Table 6. For the normal animal, the handling of the compound by the liver resembles the blood pool clearance image. The L/C ratio of about 1:1 in the normal liver implies a degree of vascular retention of the compound due to the molecular size. Normal excretion takes place via the kidneys.

Liver sections of the rats were stained with H&E, Masson Trichrome and Reticulin stains. The amount of necrosis and fibrosis were interpreted by an experienced hepatopathologist and scored according to a classification system whereby 1 was mild necrosis or fibrosis and 4 severe. The control rats, not receiving CCl₄, showed no fibrosis or necrosis, while the CCl₄-treated rats had average scores for necrosis, after 5, 7 and 9 weeks of treatment of 1.9, 1.7 and 1.4, and for fibrosis of 1.9, 1.9 and 2. 1, respectively.

These histopathological results demonstrate the relevance of this animal model to hepatic fibrosis and necrosis. The good correlation between fibrosis, necrosis and the uptake of radioactivity from the polymer under test further confirm that the present invention is suitable for diagnosis in humans.

The radiolabelled polymer complex targets fibrosis. Normal liver uptake is largely limited and the complex is characterised by fast clearance from other organs, e.g. lungs. Clearance is predominantly by the kidneys and there is low uptake by the skeleton to minimise radiation dose to the bone marrow, bearing in mind repeated patient screening. In addition, and importantly there is a degree of retention of the agent in the intact vasculature which allows time for adequate accumulation at the site of the lesion.

### EXAMPLE 5

Pyrogen and Toxicity Tests of Tc-99m PEIMP in Mice and Rabbits

Five BALB/C mice were injected intravenously, each with a dose of 0.165 mg/0.33 cc per mouse of Tc-99m 20-30 kDa PEIMP. They were observed and monitored at 24 hours and 48 hours and found to be alive and well. The injected compound was of a concentration much higher than received by the experimental animals of the project, than would be intended for clinical use, and can therefore be accepted to be non-toxic for patient application (South African Bureau of Standards Method 6:12).

Three rabbits were observed and monitored over 2 hours for body temperature fluctuations, and an average obtained for each. They subsequently received the compound injected intravenously and were observed and monitored for 2.5 hours. Differences in patterns of temperature fluctuations were recorded. They complied with requirements for non-pyrogenicity of the tested compound (Standard SABS Method 6:1992)

These results demonstrate the safety of the diagnostic agent used in this invention.

### EXAMPLE 6

A part of the theory on which the invention is based is that the calcium ions associated with collagen bind to the radiolabelled polymeric reagent in the fibrotic liver. The following experiment therefore tests the ability of Tc-labelled PEIMP to bind to collagens.

The study also compares the affinities of the two most generally used bone seeking agents, Tc-99m-PYP and Tc-99m-MDP, as well as a non-bone seeking agent, Tc-99m-DTPA (as a control) with that of Tc-99m-PEIMP (20-30 kDa) for the collagens (which were of type I and type IV). The results, are shown in Table X, show an affinity of Tc-99m-PEIMP>Tc-99m-PYP>Tc-99m-MDP>Tc-99m-DTPA for Ca²⁺-collagens type I and type IV.

All reagents were of analytical grade and all appropriate solvents and solutions were deoxygenated by purging with argon. Commercial DTPA (diethyltriaminepentaacetic acid), MDP (methylene diphosphonate) and PYP (pyrophosphate) labelling kits were labelled with Tc-99m obtained from a Mo-99/Tc-99m generator according to the manufacturers' directions. All radioactivity measurements were done in a Capintec CRC-15 dose calibrator (Capintec Inc. Pittsburgh, Pa., USA).

Due to the differences in labelling kit formulations, as well as in the diversity of the chemical composition of their active ingredients, the various final Tc-99m-labelled solutions were equalized in terms of the calculated elemental phosphorus content and diluted with tris buffer (0.01 M, pH 7.4) to a final phosphorus content of 0.025 mg/ml.

The radiolabelled complexes were analysed for radiochemical purity using instant thin-layer chromatography on silica gel impregnated glassfibre sheets as stationary phase and acetone and 0.9% sodium chloride solution as mobile phase. The radiochemical purity of the complexes was >95%.

Human collagens, type 1 (Sigma type VIII) and type IV (Sigma type VI) from human placenta were obtained from Sigma Chemical Company, St. Louis, Mo., USA.

The collagen preparations were uniformly suspended in tris buffer (pH 7.4, 0.1 M) and loaded (0.25 mg/filter) on 0.22 μm Millex-GS 25 mm diameter filter units (Millipore Products Division, Bedford Mass., USA).

The collagen preparations on the filters were saturated with Ca²⁺ ions by passing 0.5 ml (0.03 ml/min) tris buffer containing 1.12M CaCl₂ through the filters. One hour later the process was repeated and the filters were left at room temperature overnight. One hour before use another 0.5 ml CaCl₂/tris buffer was passed through the filters, whereafter the excess Ca²⁺ was washed from the filters by passing 1.0 ml Tris (0.03 ml/min) through them.

One millilitre portions of the Tc-99m-labelled radioactive substances were drawn up into one ml syringes, connected to the filters and the activity measured. The ingredients of the syringes were then slowly pushed through the collagen-loaded filters (0.03 ml/min), followed by 2 ml tris buffer, whereafter the Tc-99m activities on the filters were measured.

The activities before and after the filtration processes, as well as those adsorbed in the syringes and control filters, were corrected for decay and the percentage activity that remained on the filters were calculated. All experiments were performed in triplicate and the mean percentages of activities that remained on the filters are summarised in Table 7.

It will be seen that the Tc-99m-labelled PEIMP used in this invention out-performed the non-polymeric materials by a big margin, an unpredictable result.

All documents cited herein in the context of describing features of the invention are hereby incorporated by reference.

TABLE 1
Half lives of different technetium-99m polyethyleniminomethyl
phosphonic acid fractions in various organs of healthy baboons and in blood

| | T½ | | | | | | |
|---|---|---|---|---|---|---|---|
| Fraction | Cardiac | | | | | | Blood |
| (kDa) | Blood Pool | Liver | Kidney | Lung | Spleen | Bone | Clearance |
| 3-10 | 10 ± 1 min | 90 ± 22 min | >4 h | 10 ± 1.5 min | 75 ± 2 min | >2 h | 25 ± 4 min |
| 10-30 | 10 ± 1.5 min | 22 ± 3 min | 20 ± 3 min | 15 ± 3 min | 60 ± 9 min | >4 h | 25 ± 2 min |
| 30-50 | 6 ± 2 min | 60 ± 15 min | >4 h | 8 ± 2 min | 45 ± 8 min | >4 h | 20 ± 3 min |
| 50-100 | 12 ± 1.5 min | >4 h | >4 h | 22 ± 2 min | >4 h | >24 h ± 2 h | 45 ± 6 min |
| 100-300 | 2 h ± 0.1 h | 24 ± 1.5 h | — | 2.5 h ± 0.5 h | >24 h ± 2 h | — | 75 ± 8 min |

TABLE 2
Percentage of the injected dose of the different technetium-99m
polyethyleniminomethyl phosphonic acid fractions excreted
through the kidneys of healthy baboons at 4 h

| | % of Injected Dose |
|---|---|
| | Excreted through |
| Fraction (kDa) | the Kidneys at 4 h |
| 3-10 | 40.6 ± 5.5% |
| 10-30 | 52.8 ± 12.6% |
| 30-50 | 62.0 ± 3.9% |
| 50-100 | 28.2 ± 5.3% |
| 100-300 | 12.2 ± 5.7% |

TABLE 3
Highest percentage of the different macromolecular technetium-99m
polyethyleniminomethyl phosphonic acid fractions in various organs
of the healthy baboon in the first 4 hours

| Fraction | Organ | | | | | | |
|---|---|---|---|---|---|---|---|
| (kDa) | Background | Cardiac | Liver | Kidney | Lung | Spleen | Bone |
| 3-10 | 4 ± 0.5 | 15 ± 4.0 | 16 ± 1.0 | 36 ± 4 | 8 ± 2.3 | 10 ± 1 | 8 ± 1.00 |
| 10-30 | 12.5 ± 0.5 | 15 ± 3.0 | 20 ± 2.1 | 17.5 ± 4.0 | 12.5 ± 4.0 | 12.5 ± 1.1 | 18 ± 1.3 |
| 30-50 | 2.5 ± 0.4 | 10 ± 4.0 | 20 ± 2.0 | 40.0 ± 5.1 | 7.5 ± 2.0 | 8.0 ± 3.0 | 9 ± 0.5 |
| 50-100 | 1.3 ± 0.1 | 15 ± 4.2 | 43 ± 2.6 | 15.0 ± 2.3 | 9.0 ± 3.2 | 8.0 ± 0.5 | 7 ± 0.5 |
| 100-300 | 2.75 ± 0.3 | 30 ± 6.5 | 57 ± 9.0 | — | 17.5 ± 8.0 | 5.5 ± 2 | — |

TABLE 4
Effect of Tc-99m 20-30 kDa PEIMP in dogs

| | Normal dogs (n = 3) | | | Diseased dog (n = 1) | | |
|---|---|---|---|---|---|---|
| | T½ | T max* | % retention of ID | T½ | T max* | % retention of ID |
| Organ | (minutes) | (minutes) | at 3 hours | (minutes) | (minutes) | at 3 hours |
| Cardiac | 18 | 0 | 13 | 15 | 0 | 8.6 |
| Lungs | 10 | 0 | 15 | 10 | 0 | 13 |
| Liver | 15 | 5 | 24 | 20 | 5 | 44 |
| Left Kidney | 30 | 5 | 21 | 27 | 5 | 18 |
| Right Kidney | 35 | 5 | 24 | 27 | 5 | 16 |
| Background | 50 | 5 | 23 | 45 | 5 | 20 |

*Tmax is the time at which maximum uptake of radio-labelled polymer is reached in the particular organ listed

TABLE 5
T½ and maximum percentage activity uptake at 25 minutes
in various organs for the various rat groups

| | Controls | | 4 Weeks | | 5 Weeks | | 7 Weeks | | 9 Weeks | |
|---|---|---|---|---|---|---|---|---|---|---|
| Region | T½ | Max % | T½ | Max % | T½ | Max % | T½ | Max % | T½ | Max % |
| Cardiac | 4 min | 22 | 3.9 min | 21 | 2.6 min | 27 | 2 min | 26 | 2.5 min | 28 |
| Liver | 4 min | 20 | | 31 | | 35 | | 44 | | 53.9 |
| Kidney | 6 min | 37 | 10 min | 25 | 10 min | 30 | 5 min | 26 | 7.5 min | 28 |

TABLE 6
Liver/cardiac blood pool ratios in rats

| | | GROUP 1 | GROUP 2 | GROUP 3 | |
|---|---|---|---|---|---|
| | MINS | (5 weeks) | (7 weeks) | (9 weeks) | CONTROLS |
| 1 | 2 | 1.26 | 2.03 | 1.62 | 0.84 |
| 2 | 5 | 1.91 | 3.08 | 2.48 | 0.82 |
| 3 | 10 | 2.49 | 4 | 3.37 | 0.84 |
| 4 | 15 | 2.67 | 4.45 | 3.85 | 0.84 |
| 5 | 20 | | 4.76 | 4.03 | 0.84 |
| 6 | 25 | | 5.05 | 4.4 | 0.87 |

TABLE 7
Percentage of radioactivity that remained
on the collagen-loaded filters

| | Radioactive Substances | | | |
|---|---|---|---|---|
| | Tc-99m- | Tc-99m- | Tc-99m- | Tc-99m- |
| Collagen | DTPA | MDP | PYP | PEIMP |
| Type-I | 1.78% | 6.51% | 6.80% | 19.95% |
| Type-IV | 1.22% | 0.81% | 1.72% | 4.37% |

1. A method of in vivo diagnosis of hepatic fibrosis or necrosis in a patent, which comprises administering parenterally to the patient an effective amount of a complex of (1) a polymer having free phosphonate groups positioned to provide polydentate ligands capable of forming stable complexes with radionuclide ions and capable of binding calcium ions and (2) radionuclide ions which are non-toxic in the diagnosis, determining the degree of the retention of the radionuclide within the liver of the patient, relative to that of normal patients, and from the result of this determination making a diagnosis as to whether the patient is suffering from hepatic fibrosis or necrosis or the extent thereof, said polymer having a molecular weight which enables uptake into the liver, retention by a diseased (fibrotic or necrotic) liver for sufficient time reliably to count the radioactivity in the liver so that it can be compared with a normal liver and sufficiently rapid clearance by the kidneys to prevent long-term harm to the patient.

2. A method according to claim 1, wherein the polymer has a relative molecular mass (excluding radionuclide ions) of from 10 to 30 kDa.

3. A method according to claim 2, wherein the polymer has a relative molecular mass of from 20 to 30 kDa.

4. A method according to claim 1, 2 or 3, wherein the polymer is a dendrimer, having a branch upon branch chain structure.

5. A method according to claim 1, 2, 3 or 4, wherein the polymer is a polyamine.

6. A method according to claim 5, wherein the polyamine is an ethylene imine polymer.

7. A method according to claim 6, wherein the ethylene imine polymer is a homopolymer.

8. A method according to claim 1, 2, 3, 4, 5, 6 or 7, wherein the phosphonate groups are alkylphosphonates of formula —R—(PO(OH)₂)ₙ wherein n is 1 or 2 and R represents a divalent alkylene group of 1 to 4 carbon atoms, which may be hydroxy-substituted, or its trivalent counterpart, according to whether n=1 or 2.

9. A method according to claim 8, wherein the alkylphosphonate is a methylphosphonate.

10. A method according to claim 9, wherein the polymer is polyethyleniminomethyl phosphonate of relative molecular mass (excluding radionuclide ions) of from 20 to 30 kDa.

11. A method according to claim 1, 2, 3, 4, 5, 6, 7, 8, 9 or 10, wherein the complex is prepared from a reducible salt of the radionuclide and a reducing agent.

12. A method according to claim 1, 2, 3, 4, 5, 6, 7, 8, 9, 10 or 11, wherein the radionuclide is a gamma ray-emitter and the extent of retention of the radionuclide is determined by gamma ray scintigraphy.

13. A method according to claim 12, wherein the radionuclide is technetium-99m, the symbol “m” denoting that the isotope is metastable.

14. A method according to claim 13, wherein the complex is prepared from sodium pertechnetate, a stannous salt as reducing agent and the polymer.

15. A method according to claim 1, 2, 3, 4, 5, 6, 7, 8, 9, 10, 11, 12, 13 or 14, wherein the complex is injected into the patient as a sterile aqueous solution or dispersion.

16. Use of a polymer having free phosphonate groups positioned to provide a polydentate ligand capable of forming stable complexes with radionuclide salts and capable of binding calcium ions, in the preparation of a formulation thereof as a complex with radionuclide ions, for use in the in vivo diagnosis of hepatic fibrosis or necrosis or the extent thereof, the radionuclide ions being non-toxic in such diagnosis and said polymer having a molecular weight which enables uptake into the liver, retention by a diseased (fibrotic or necrotic) liver for sufficient time reliably to count the radioactivity in the liver so that it can be compared with a normal liver and sufficiently rapid clearance by the kidneys to prevent long-term harm to the patient.

17. Use according to claim 16, wherein the polymer is as defined in any one of claims 2 to 10.

18. Use according to claim 16 or 17, wherein the radionuclide or complex is as defined in claim 11, 12, 13 or 14.

19. Use according to claim 16, 17 or 18, wherein the formulation is a sterile injectible, aqueous solution or dispersion.
